(19) United States
(12) Patent Application Publication
(10) Pub. No.: US 2024/0282413 A1
(43) Pub. Date: Aug. 22, 2024

(54) SIMILAR CLINICAL TRIAL SEARCH SUPPORT SYSTEM AND SIMILAR CLINICAL TRIAL SEARCH SUPPORT METHOD

(71) Applicant: Hitachi, Ltd., Tokyo (JP)

(72) Inventors: Hiroko OTAKI, Tokyo (JP); Kunihiko KIDO, Tokyo (JP)

(21) Appl. No.: 18/426,440

(22) Filed: Jan. 30, 2024

(30) Foreign Application Priority Data
Feb. 17, 2023 (JP) 2023-023747

(51) Int. Cl. G16H 10/20; G16H 50/70

(52) U.S. Cl. CPC G16H 10/20; G16H 50/70

(57) ABSTRACT

A similar clinical trial search support system stores clinical trial information related to a plurality of clinical trials conducted in the past, and clinical trial item information that associates a clinical trial item in which a setting item of each clinical trial is described in a character string, performs a search based on a first search item to acquire the identification information of one or more clinical trials as a first search result, adds a second search item to perform a search, so as to acquire identification information of the one or more clinical trials as a second search result, acquires a plurality of the clinical trial items corresponding to the first search result from the clinical trial item information, clusters the plurality of acquired clinical trial items to generate a plurality of clusters, and calculates, for each cluster, a ratio of the number of the clinical trial items.

### CLAIM OF PRIORITY

The present application claims priority from Japanese patent application JP 2023-023747 filed on Feb. 17, 2023, the content of which is hereby incorporated by reference into this application.

### BACKGROUND OF THE INVENTION

### 1. Field of the Invention

The present invention relates to a technique for supporting clinical trial plan formulation.

### 2. Description of Related Art

As a technique for supporting a plan of a clinical test, for example, JP2019-56974A (PTL 1) is disclosed. PTL 1 describes “an information processing program causing a computer to execute a process of: referring to a storage unit that stores a plurality of pieces of clinical trial information each associating identification information for identifying a clinical trial with a clinical trial item in the clinical trial, and acquiring the clinical trial information corresponding to the identification information for identifying a target of the clinical trial; specifying, using a search item of the same category for the storage unit, the identification information of past clinical trial information that includes a clinical trial item equivalent to a clinical trial item that corresponds to the search item in the acquired clinical trial information, from the storage unit; referring to a storage unit that stores setting information associating the identification information for identifying a clinical trial with an index set in a past clinical trial, and specifying an index that corresponds to the identification information of the specified past clinical trial information; and outputting an index that does not match the index set for the target of the clinical trial from the specified indices”.

### CITATION LIST

### Patent Literature

PTL 1: JP2019-56974A

### SUMMARY OF THE INVENTION

In a process of development of a new drug, a basic study for selecting a highly likely compound from new drug candidates is performed, and then a non-clinical test for examining a pharmacological action is performed using animals. After that, a clinical test is performed, a result of the clinical test is submitted to Ministry of Health, Labour and Welfare, so as to be subjected to an approval examination, and when production is approved, the new drug can be released.

The clinical test is a study performed to examine effectiveness and safety of the new drug for humans. It is necessary to secure a sufficient number of subjects to exhibit statistical significance in relation to the effectiveness and safety and acquire test data under high quality control, and reliability of the test data and ethical considerations for subjects are also required. Therefore, in order to perform a clinical test, a high cost is required, and if the clinical test cannot be appropriately conducted and the development of the new drug fails, a pharmaceutical company has a loss of the high cost. If the appropriate clinical test cannot be conducted, the clinical test is prolonged, and the release of the new drug is delayed, which has a significant impact on patients.

Therefore, pharmaceutical companies need to carefully consider a content of clinical tests to be conducted and create plans, so that highly reliable test data that is useful for developing new drugs can be obtained. In addition, it is necessary to create a clinical trial conduct plan when the clinical test is conducted in a medical facility such as a hospital, and deliver the clinical trial conduct plan to Ministry of Health, Labour and Welfare.

In creating a plan of clinical tests, information on clinical tests planned in the past is fairly useful, and it is common to create a plan by referring to similar clinical tests planned in the past. Further, with reference to guidelines related to clinical tests, guidelines related to treatments, and laws of restrictions such as a pharmaceutical law, a plan is created such that the effectiveness and safety can be investigated by a method in scientific and ethical consideration.

A database is known in the related art in which information on clinical tests is registered so that information on a past clinical test plan can be effectively used. Examples include public databases such as the PubMed service, which allows users to search for abstracts of academic papers on the Internet, and Internet site ClinicalTrials. gov for registering a clinical trial conduct plan of a clinical test.

In general, using such a database, a clinical trial conduct plan for a disease to be treated of a drug to be subjected to a clinical test and a drug having an action mechanism similar to that of the drug is comprehensively investigated, and test conditions are set according to a purpose of the clinical test.

For example, a setting of a new clinical trial is determined with reference to the clinical trial items set in the past clinical trial. Specifically, the database is searched using a disease subjected to be interested (for example, a disease targeted by a development drug of the company) as a keyword, and a list of clinical trials including the keyword is displayed. Then, a search keyword is set in more detail, and clinical trials similar to adaptation of the development drug are narrowed down from the clinical trial of the target disease.

At this time, in the related art, it has been determined whether the contents of the items of the clinical trial set in the clinical trial similar to the adaptation of the development drug are items specific to the clinical trial similar to the adaptation or items common to the entire target disease, and whether the items of the searched clinical trial are to be adopted for the new clinical trial while analyzing a tendency. However, since the description of contents of the clinical trial items is free description, it takes time to determine identity of the contents of the clinical trial items when the number of clinical trials increases, and it is difficult to perform a polygonal analysis in which analysis is performed while variously changing the search conditions.

In order to solve at least one of the above-described problems, the invention provides a similar clinical trial search support system including a processing unit and a storage unit. The storage unit stores clinical trial information that associates identification information of each of a plurality of clinical trials conducted in the past with a content of the clinical trial, and clinical trial item information that associates the identification information of the clinical trial with one or more clinical trial items in which a setting item of the clinical trial is described in a character string. The processing unit searches the clinical trial information based on a first search item to acquire the identification information of one or more clinical trials as a first search result, adds a second search item to the first search item to search the clinical trial information, so as to acquire identification information of the one or more clinical trials as a second search result, acquires a plurality of the clinical trial items corresponding to the identification information of the one or more clinical trials acquired as the first search result from the clinical trial item information, clusters the plurality of acquired clinical trial items to generate a plurality of clusters, calculates, for each cluster, a ratio of the number of the clinical trial items corresponding to the identification information of the clinical trial acquired as the second search result in the clinical trial items classified into the cluster, and outputs information for specifying the cluster, and the ratio calculated for the cluster.

According to an aspect of the invention, a polygonal analysis of the past clinical trial information focusing on similarity to adaptation of a development drug is supported.

The problems, configurations, and effects other than those described above will become apparent according to the following description of embodiments.

### BRIEF DESCRIPTION OF THE DRAWINGS

FIG. 1 is a block diagram showing a configuration of a similar clinical trial search support system according to an embodiment of the invention.

FIG. 2 is a sequence diagram showing a process executed by the similar clinical trial search support system according to the embodiment of the invention.

FIG. 3 is a diagram showing a clinical trial information storage unit held by the similar clinical trial search support system according to the embodiment of the invention.

FIG. 4 is a diagram showing a clinical trial item storage unit held by the similar clinical trial search support system according to the embodiment of the invention.

FIG. 5 is a diagram showing a clinical trial item cluster storage unit held by the similar clinical trial search support system according to the embodiment of the invention.

FIG. 6 is a diagram showing a search history storage unit held by the similar clinical trial search support system according to the embodiment of the invention.

FIG. 7 is a diagram showing a medical organism term storage unit held by the similar clinical trial search support system according to the embodiment of the invention.

FIG. 8 is a flowchart showing a ratio calculation process executed by the similar clinical trial search support system according to the embodiment of the invention.

FIG. 9 is a flowchart showing a clustering process executed by the similar clinical trial search support system according to the embodiment of the invention.

FIG. 10 is a diagram showing subtype ratios output by the similar clinical trial search support system according to the embodiment of the invention.

FIG. 11 is a diagram showing a classification of clinical trials output by the similar clinical trial search support system according to the embodiment of the invention.

### DESCRIPTION OF EMBODIMENTS

Hereinafter, a similar clinical trial search support system according to a preferred embodiment of the invention will be described in detail with reference to the drawings.

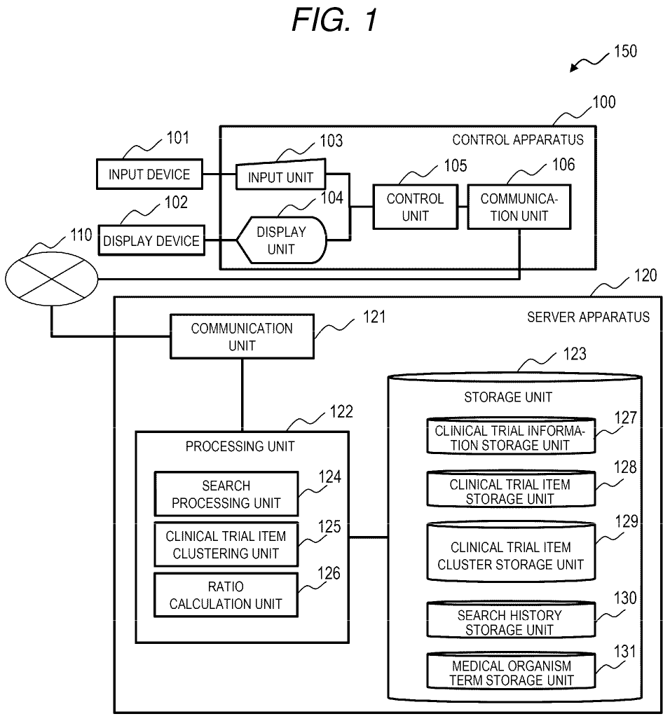

FIG. 1 is a block diagram showing a configuration of a similar clinical trial search support system 150 according to an embodiment of the invention.

The similar clinical trial search support system 150 according to the embodiment includes a server apparatus 120 and a control apparatus 100. The server apparatus 120 is a computer system including a communication unit 121, a processing unit 122, and a storage unit 123. The communication unit 121 is an interface that transmits and receives data to and from another device (the control apparatus 100 in the example in FIG. 1) connected to the server apparatus 120 via a network 110. The processing unit 122 executes various processes according to programs. In the embodiment, the processing unit 122 includes a processor that implements processes of a search processing unit 124, a clinical trial item clustering unit 125, and a ratio calculation unit 126. A program for implementing the above-described units may be stored in, for example, a memory in the processing unit 122 or may be stored in the storage unit 123.

The storage unit 123 is a storage device such as a hard disk drive (HDD) or a solid state drive (SSD), and stores information referred by the processing unit 122 to execute processes and information generated by the processes. The storage unit 123 according to the embodiment includes a clinical trial information storage unit 127, a clinical trial item storage unit 128, a clinical trial item cluster storage unit 129, a search history storage unit 130, and a medical organism term storage unit 131. Information stored in these units will be described later.

The control apparatus 100 is a terminal apparatus that transmits a request for a process to the server apparatus 120 according to an input of a user, receives a result of the process according to the request from the server apparatus 120, and displays the result to the user. The control apparatus 100 includes an input unit 103, a display unit 104, a control unit 105, and a communication unit 106. The input unit 103 is connected to an input device 101 and receives an input from a user via the input device 101. The control unit 105 transmits a processing request to the server apparatus 120 via the communication unit 106 according to the input from the user. In addition, when a processing result is received via the communication unit 106, a display of the processing result by the display unit 104 is controlled. The display unit 104 is connected to a display device 102 and displays the processing result via the display device 102. The communication unit 106 transmits and receives data to and from the server apparatus 120 via the network 110.

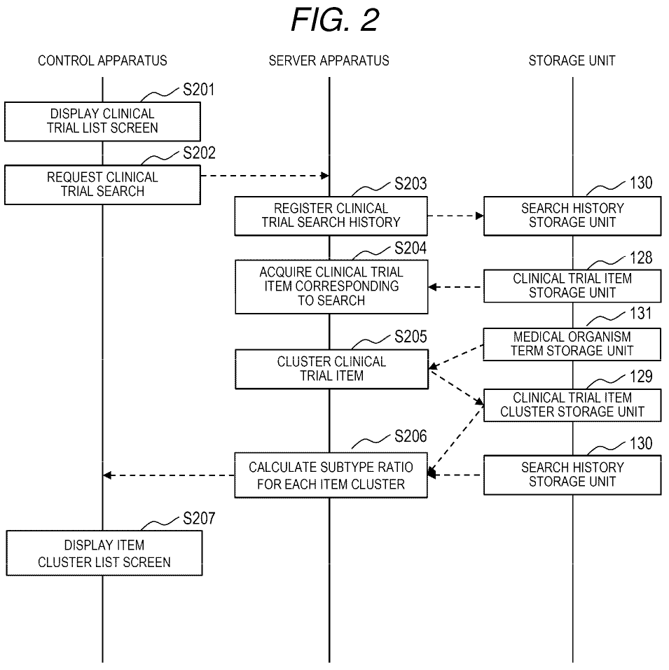

FIG. 2 is a sequence diagram showing a process executed by the similar clinical trial search support system 150 according to the embodiment of the invention.

First, the control apparatus 100 displays a clinical trial list screen (step S201). For example, the control apparatus 100 may communicate with the server apparatus 120 to acquire information stored in the clinical trial information storage unit 127, and display a list of information related to clinical trials via the display device 102.

Next, the control apparatus 100 sends a clinical trial search request to the server apparatus 120 (step S202). For example, when the user inputs a disease, which the user is interested in, to the control apparatus 100 via the input device 101, the control apparatus 100 sends a clinical trial search request for the disease to the server apparatus 120. Here, the disease that the user is interested in is, for example, a disease targeted by a drug developed by the user.

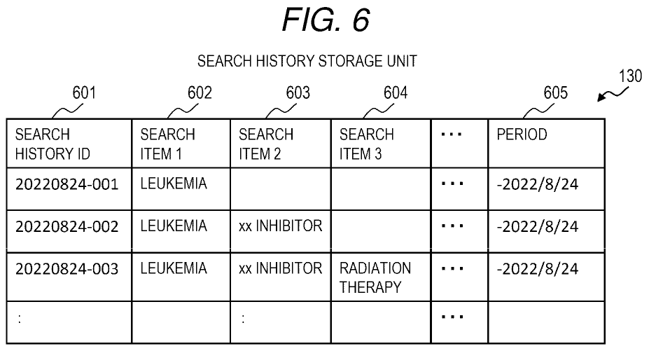

When the server apparatus 120 receives the clinical trial search request from the control apparatus 100, the server apparatus 120 registers a clinical trial search history in the search history storage unit 130 (step S203). Details thereof will be described later with reference to FIG. 6.

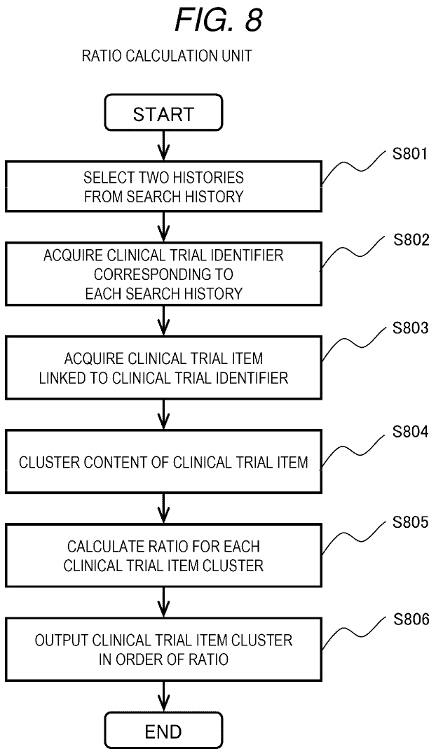

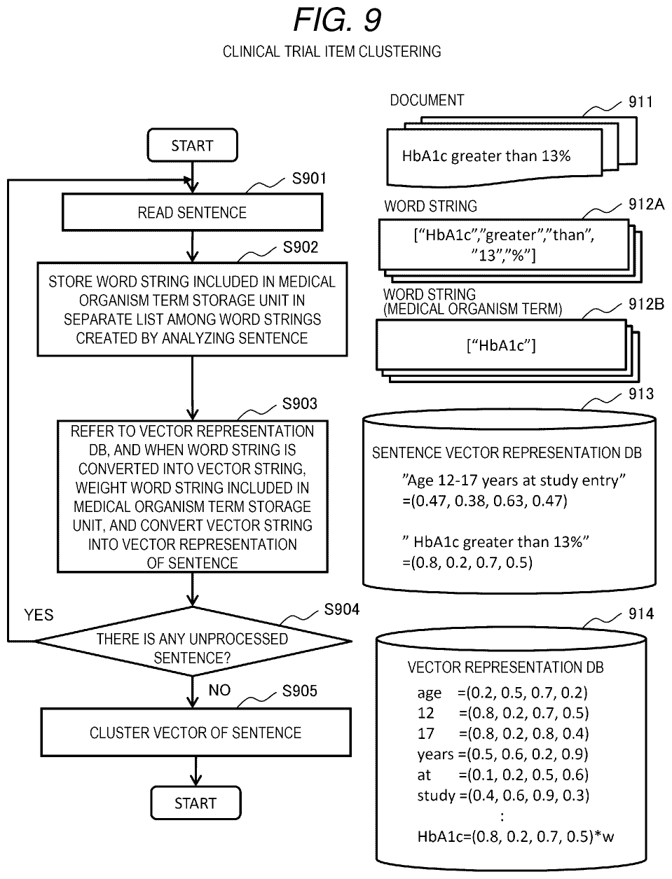

Next, the server apparatus 120 searches for clinical trial items stored in the clinical trial item storage unit 128 according to the received clinical trial search request, acquires the clinical trial items corresponding to a search keyword (step S204), and clusters the acquired clinical trial items (step S205). At this time, the server apparatus 120 performs clustering by referring to the medical organism term storage unit 131, and stores a clustering result in the clinical trial item cluster storage unit 129. Then, the server apparatus 120 calculates a subtype ratio for each cluster (step S206). The subtype ratio is a ratio of clinical trial items, in the clinical trial items classified into clusters, acquired from the clinical trial items related to the clinical trials that are obtained by adding the search keywords and performing a narrowed-down search. Details of Steps S204 to S206 will be described later with reference to FIGS. 8 and 9.

The server apparatus 120 transmits the clustering result and the calculated subtype ratio to the control apparatus 100. The control apparatus 100 displays a cluster list screen of the clinical trial items based on the subtype ratio (step S207).

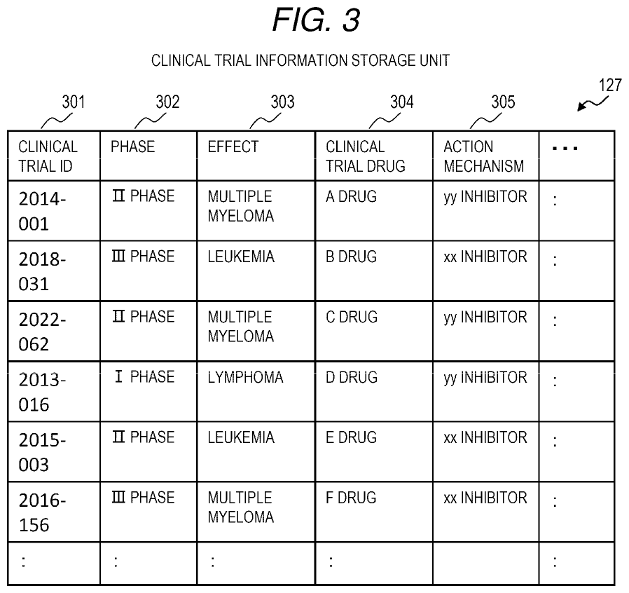

FIG. 3 is a diagram showing the clinical trial information storage unit 127 held by the similar clinical trial search support system 150 according to the embodiment of the invention.

The clinical trial information storage unit 127 stores information related to clinical trials conducted in the past. For example, the clinical trial information storage unit 127 includes a clinical trial ID 301, a phase 302, an effect 303, a clinical trial drug 304, and an action mechanism 305. The clinical trial ID 301 is an identifier of each clinical trial. The phase 302 indicates a phase of a conducted clinical trial. The clinical trial drug 304, the effect 303, and the action mechanism 305 respectively indicate information for identifying a drug targeted in the clinical trial, the effect of the drug, and the action mechanism of the drug.

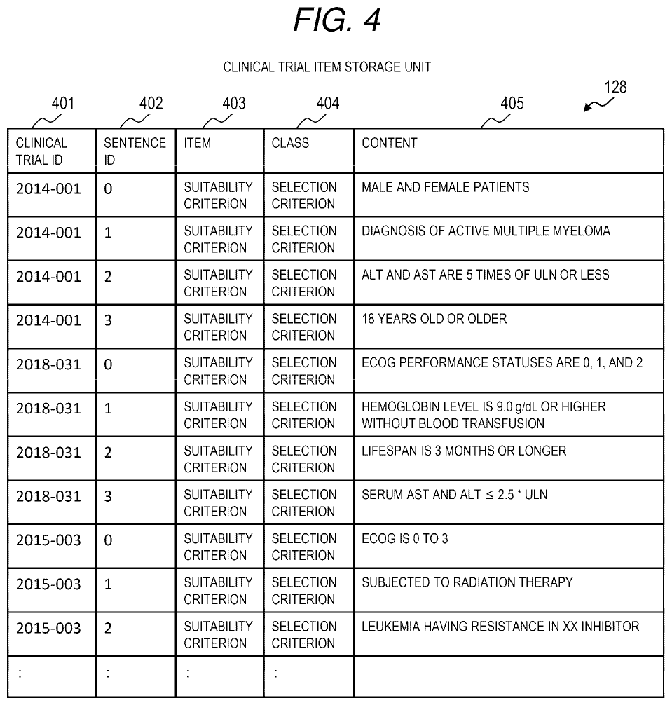

FIG. 4 is a diagram showing the clinical trial item storage unit 128 held by the similar clinical trial search support system 150 according to the embodiment of the invention.

The clinical trial item storage unit 128 stores information related to the items set in the clinical trials conducted in the past. For example, the clinical trial item storage unit 128 includes a clinical trial ID 401, a sentence ID 402, an item 403, a class 404, and a content 405. The clinical trial ID 401 is an identifier of each clinical trial, and corresponds to the clinical trial ID 301 in FIG. 3. The sentence ID 402 is an identifier of an item set in each clinical trial. The item 403 and the class 404 indicate a type, property, and the like of each set item. The content 405 is a content of each set item, and includes text information such as a sentence.

In the example in FIG. 4, four clinical trial items are set in the clinical trial identified by a clinical trial ID “2014-001”. In any of the items, the item 403 and the class 404 are a “suitability criterion” and a “selection criterion”, respectively. This indicates that each item is a criterion for selecting a person who is suitable as the clinical trial target. The contents 405 of the four clinical trial items identified by the sentence IDs 402 of “0” to “3” of the clinical trials with the clinical trial ID 401 of “2014-001” are “male and female patients”, “diagnosis of active multiple myeloma”, “ALT and AST are 5 times of ULN or less”, and “18 years old or older”. This indicates that, as a subject (that is, a subject to which a drug targeted in the clinical trial is administered) of the clinical trial, a person with ALT and AST of 5 times of the ULN or less, who has been diagnosed for the active multiple myeloma at 18 years old or older, has been set regardless of a gender.

Similarly, the clinical trial item storage unit 128 stores information indicating one or more clinical trial items set for other clinical trials.

Although FIG. 4 shows only the suitability criterion as an example, the clinical trial item storage unit 128 may store clinical trial items having values of the item 403 and the class 404 different from those described above, such as an “exclusion criterion”, which is a criterion for excluding the clinical trial item from a suitable person targeted in a clinical trial.

Information describing the subject to which the drug is administered, for example, information indicating under what condition is the drug to be administered to a person is also referred to as adaptation.

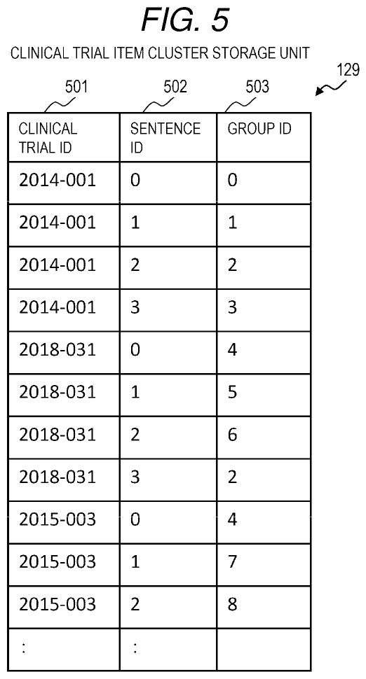

FIG. 5 is a diagram showing the clinical trial item cluster storage unit 129 held by the similar clinical trial search support system 150 according to the embodiment of the invention.

The clinical trial item cluster storage unit 129 stores the clustering result (step S205). For example, the clinical trial item cluster storage unit 129 includes a clinical trial ID 501, a sentence ID 502, and a group ID 503. The clinical trial ID 501 and the sentence ID 502 correspond to the clinical trial ID 401 and the sentence ID 402 shown in FIG. 4, respectively, and are the identifier of the clinical trial and the identifier of the item set in each clinical trial. The group ID 503 indicates a group (that is, a cluster) into which each item is classified by clustering.

In the examples in FIGS. 4 and 5, the item that “ALT and AST are 5 times of ULN or less” of the clinical trial with the clinical trial ID “2014-001” and an item that “serum AST and ALT ≤2.5*ULN” of the clinical trial with a clinical trial ID “2018-031” are classified into the same group. Also, an item “ECOG performance statuses are 0, 1, and 2” of the clinical trial with the clinical trial ID “2018-031” and an item “ECOG is 0 to 3” of the clinical trial with the clinical trial ID “2015-003” are classified into the same group.

FIG. 6 is a diagram showing the search history storage unit 130 held by the similar clinical trial search support system 150 according to the embodiment of the invention.

The search history storage unit 130 stores a history of searches performed in the past. For example, the search history storage unit 130 includes a search history ID 601, search items 1_602 to 3_604, and a period 605. The search history ID 601 is an identifier of the performed search. The search items 1_602 to 3_604 indicate items designated as keywords in the search. The period 605 is information designating a range of a period in which a clinical trial as a search target was performed.

The example in FIG. 6 shows a history in which, for clinical trial items related to the clinical trials performed up to Aug. 24, 2022, first, a search was performed using “leukemia” as a keyword, next, a narrowed-down search (that is, AND search) was performed by adding a keyword “xx inhibitor” to “leukemia”, and next, a narrowed-down search was performed by further adding a keyword “radiation therapy” to “leukemia” and “xx inhibitor”.

Although not shown in FIG. 6, in practice, more keywords may be added to perform a narrowed-down search, and thus the search history storage unit 130 may further include an item that holds more keywords such as search items 4 and 5.

The clinical trial search request transmitted in step S202 in FIG. 2 includes information designating the search keyword, and in step S203, the server apparatus 120 adds a new record to the search history storage unit 130 and registers the received search keyword in the search item 1_602 or the like.

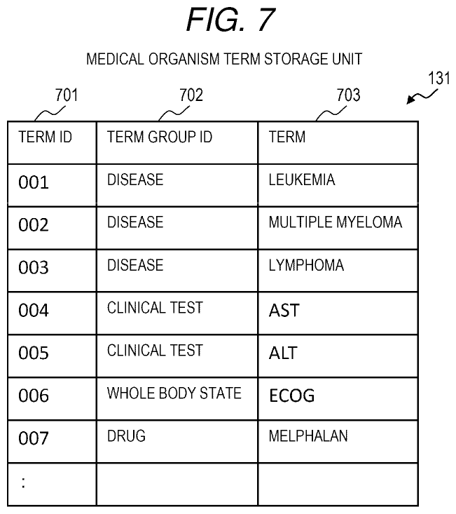

FIG. 7 is a diagram showing the medical organism term storage unit 131 held by the similar clinical trial search support system 150 according to the embodiment of the invention.

The medical organism term storage unit 131 stores at least one of medical terms and organism terms assumed to be included in the clinical trial items. For example, the medical organism term storage unit 131 includes a term ID 701, a term group ID 702, and a term 703. The term ID 701 is an identifier of each term. The term group ID 702 is an identifier of a group to which each term belongs. The term 703 is a term thereof.

In the example in FIG. 7, “leukemia”, “multiple myeloma”, “lymphoma”, “AST”, “ALT”, “ECOG”, and “melphalan” are registered as the term 703. In this example, the terms of “leukemia”, “multiple myeloma”, and “lymphoma” belong to a term group “disease”. The terms of “AST” and “ALT” belong to a term group “clinical test”. The term of “ECOG” belongs to a term group “whole body state”. The term of “melphalan” belongs to a term group “drug”.

The medical organism term storage unit 131 may include information indicating a hierarchical relationship between terms. For example, “leukemia”, “multiple myeloma”, and “lymphoma” are subordinate concepts of “disease”. When the medical organism term storage unit 131 includes a term corresponding to a subordinate concept of a term such as “leukemia” or “multiple myeloma”, the medical organism term storage unit 131 may further include information that associates a term of a dominant concept with a term of a subordinate concept.

FIG. 8 is a flowchart showing the ratio calculation process executed by the similar clinical trial search support system 150 according to the embodiment of the invention.

The flowchart of FIG. 8 shows the process executed in steps S204 to S206 in FIG. 2 in detail.

First, the search processing unit 124 selects two search histories from the search history storage unit 130 (step S801). This is selected based on the search keyword designated by the user upon the clinical trial search request in step S202 in FIG. 2. For example, when “leukemia” is designated as the search keyword first and then “xx inhibitor” is designated as an additional keyword for a narrowed-down search, first and second rows in FIG. 6 are selected.

Next, the search processing unit 124 acquires a clinical trial identifier corresponding to each search history (step S802). For example, when the first and second rows in FIG. 6 are selected in step S801 as described above, the search processing unit 124 first searches the clinical trial information storage unit 127 using “leukemia” as a keyword. In the example in FIG. 3, the clinical trial ID “2018-031” on a second row and the clinical trial ID “2015-003” on a fifth row both include “leukemia” as the effect 303, and thus the clinical trial IDs are acquired. Further, the search processing unit 124 performs an AND search using “leukemia” and “xx inhibitor” as the keywords. The clinical trial ID “2018-031” and the clinical trial ID “2015-003” on the fifth row described above both include “xx inhibitor” as the action mechanism 305, and thus the clinical trial IDs are acquired. If a clinical trial including “leukemia” but not including “xx inhibitor” is stored in the clinical trial information storage unit 127, the clinical trial ID is acquired as a search result using “leukemia” as the keyword, but is not acquired as an AND search g “leukemia” and “xx inhibitor” as the keywords.

Next, the search processing unit 124 acquires the clinical trial item corresponding to the clinical trial identifier (that is, the clinical trial ID) acquired in step S802 (step S803). For example, when the clinical trial IDs “2018-031” and “2015-003” are acquired as described above, at least four clinical trial items having the clinical trial ID 401 of “2018-031” and the sentence IDs 402 of “0” to “3” and four clinical trial items having the clinical trial ID 401 of “2015-003” and the sentence IDs 402 of “0” to “2” in the clinical trial item storage unit 128 shown in FIG. 4 are acquired in step S803.

Next, the clinical trial item clustering unit 125 clusters the contents 405 of the clinical trial items acquired at step S803 (step S804). Details of this clustering will be described later with reference to FIG. 9 and the like.

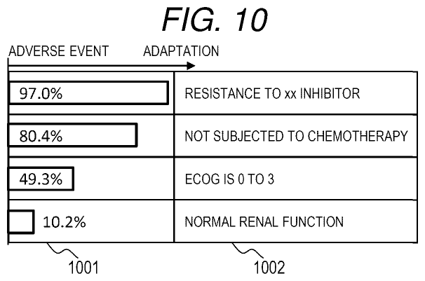

Next, the ratio calculation unit 126 calculates the subtype ratio for each cluster classified in step S806 (step S805), and outputs the clinical trial items in the order based on the calculated ratios (step S806). Details of these processes will be described later with reference to FIG. 10 and the like.

FIG. 9 is a flowchart showing a clustering process executed by the similar clinical trial search support system 150 according to the embodiment of the invention.

The flowchart of FIG. 9 shows the process executed in step S804 in FIG. 8 in detail.

First, the clinical trial item clustering unit 125 reads a sentence for which a vector of the sentence is not generated yet from the contents 405 (shown as a document 911 in FIG. 9) of all the clinical trial items acquired in step S803 (step S901). Next, the clinical trial item clustering unit 125 analyzes the read sentences to generate a word string 902A (step S902). At this time, the clinical trial item clustering unit 125 stores words included in the medical organism term storage unit 131 among words included in the word string in a medical organism term word string 912B separately from the word string 902A.

For example, when a sentence “HbAlc greater than 13%” is read in step S901, “HbAlc”, “greater”, “than”, “13”, and “%” are generated as a word string. Among these words, “HbAlc” included in the medical organism term storage unit 131 is stored in the medical organism term word string 912B, and the other words are stored in the word string 912A.

Similarly, when a sentence “Age 12-17 years at study entry” is read in step S901, “age”, “12”, “17”, “at”, “study”, and “entry” are generated as the word string 912A. The generated word strings 912A and 912B are stored in the storage unit 123.

The clinical trial item clustering unit 125 then refers to a vector representation database (DB) 914 stored in the storage unit 123, converts the word string into a vector string, and converts the converted into vector string a vector representation of the sentence (step S903). At this time, the clinical trial item clustering unit 125 weights the vector string of words stored in the medical organism term word string 912B, and converts the vector string into a vector representation of the sentence.

For example, when the terms of “age”, “12”, “17”, “at”, “study”, and “entry” are represented by vectors such as (0.2, 0.5, 0.7, 0.2), (0.8, 0.2, 0.7, 0.5), (0.8, 0.2, 0.8, 0.4), (0.5, 0.6, 0.2, 0.9), (0.1, 0.2, 0.5, 0.6), and (0.4, 0.6, 0.9, 0.3), respectively, a vector (0.47, 0.38, 0.63, 0.47) obtained by averaging these values of the vectors may be generated as the vector representation of the sentence “Age 12-17 years at study entry”.

A vector representation is also generated in the same manner as described above for the sentence “HbAlc greater than 13%”. However, in this case, since the word “HbAlc” is stored in the medical organism term word string 912B, the vector representation of the sentence “HbAlc greater than 13%” is generated after multiplying a vector representing “HbAlc” by a predetermined weight coefficient. The generated vector representation of the sentence is stored in the storage unit 123 as a sentence vector representation DB 913.

When the medical organism term storage unit 131 includes the information that associates the term of the dominant concept with the term of the subordinate concept, the weight coefficient of each term may be set such that the weight coefficient corresponding to the term of the subordinate concept is larger than the weight coefficient corresponding to the term of the dominant concept.

Next, the clinical trial item clustering unit 125 determines whether there is any unprocessed sentence (step S904), and if there is any unprocessed sentence (step S904: YES), the process returns to step S901 and the process in step S901 and thereafter is executed for the unprocessed sentence. When there is no unprocessed sentence (step S904: NO), the clinical trial item clustering unit 125 clusters the vectors of the converted sentences (step S905). As a clustering method, any method such as a K-means method or hierarchical clustering can be used. The number of clusters may be determined in advance or may be designated by the user.

FIG. 10 is a diagram showing subtype ratios output by the similar clinical trial search support system 150 according to the embodiment of the invention.

In the example in FIG. 10, a clinical trial item content 1002 and a subtype ratio 1001 calculated for the clinical trial item content 1002 are displayed in descending order of a value of the subtype ratio 1001. Each row of the table of FIG. 10 corresponds to a respective one of clusters generated by the clinical trial item clustering unit 125. The subtype ratio of the cluster calculated by the ratio calculation unit 126 may be displayed in the subtype ratio 1001 of the row, and a representative value (for example, the value of the content 405 of any of the clinical trial items of each cluster) of the content 405 of the clinical trial item classified into each cluster may be displayed in the content 1002 of the clinical trial item.

Here, a specific example of the above-described process will be described with reference to FIGS. 3 to 9. Here, as an example, a case will be described in which a user develops a drug related to leukemia, assumes a “patient of leukemia having resistance in xx inhibitor”, a “patient subjected to radiation therapy”, or the like as a target to which the drug is administered, and attempts to conduct a clinical trial of the drug.

In this case, the user selects, for example, the search histories “20220824-001” and “20220824-002” stored in the search history storage unit 130 (step S801). The former includes “leukemia” as the search item, and the latter includes “leukemia” and “xx inhibitor” as the search items.

In step S802, as described above, the clinical trial IDs “2018-031” and “2015-003” are acquired as the clinical trial identifiers corresponding to the search history “20220824-001”, and the clinical trial IDs “2018-031” and “2015-003” are also acquired as the clinical trial identifiers corresponding to the search history “20220824-002”.

Here, if the clinical trial information storage unit 127 stores a clinical trial that includes “leukemia” as the effect 303 but does not include “xx inhibitor” as the action mechanism 305, the clinical trial ID thereof is acquired as the clinical trial ID corresponding to the search history “20220824-001” but is not acquired as the clinical trial identifier corresponding to the search history “20220824-002”.

Alternatively, if the effect 303 corresponding to the clinical trial ID “2015-003” includes “leukemia” but the action mechanism 305 does not include “xx inhibitor”, the clinical trial ID “2015-003” is acquired as the clinical trial ID corresponding to the search history “20220824-001” but is not acquired as the clinical trial identifier corresponding to the search history “20220824-002”.

In step S803, at least the clinical trial items in the fifth to eleventh rows corresponding to the clinical trial IDs “2018-031” and “2015-003” are acquired from the clinical trial items stored in the clinical trial item storage unit 128 shown in FIG. 4. Then, in step S804, character strings of the contents 405 of the clinical trial items are clustered. As a result, for example, “ECOG performance statuses are 0, 1, and 2” in the fifth row and “ECOG is 0 to 3” in the ninth row are classified into the same cluster (for example, the cluster with the group ID of “4” shown in FIG. 5).

At this time, for example, when the word “ECOG” is stored in the medical organism term storage unit 131, a vector of the word is multiplied by a predetermined weight coefficient. Accordingly, sentences including the same (or similar) medical organism terms are likely to be classified into the same cluster. Further, s described above, when the weight coefficient corresponding to the term of the subordinate concept is larger than the weight coefficient corresponding to the term of the dominant concept, sentences commonly including medical organism terms corresponding to the subordinate concept are likely to be classified into the same cluster. Accordingly, it is possible to perform appropriate clustering according to a purpose of searching for similar clinical trials.

In step S805, the ratio is calculated for each cluster. For example, when a cluster including only two character strings of “ECOG performance statuses are 0, 1, and 2” and “ECOG is 0 to 3” is generated, the former character string is the content of the clinical trial item of the clinical trial with the clinical trial ID of “2018-031”, and the latter character string is the content of the clinical trial item of the clinical trial with the clinical trial ID of “2015-003”. Each of these clinical trial IDs is obtained by the search using “leukemia” as the keyword, and is also obtained as a result of the AND search using “leukemia” and “xx inhibitor” as the keywords.

That is, in the two character strings included in the cluster, the number of character strings included in the clinical trial item obtained by the search using “leukemia” as the keyword is 2, the number of character strings included in the clinical trial item obtained by the narrowed-down search in which the keyword of “xx inhibitor” is further added to “leukemia” is 2, and the subtype ratio is 2/2=1 (that is, 100%).

If the action mechanism 305 of the clinical trial with the clinical trial ID of “2015-003” does not include “ECOG”, the clinical trial ID is not obtained as the result of the AND search using “leukemia” and “xx inhibitor” as the keywords. In this case, in the two character strings included in the cluster, the number of character strings included in the clinical trial item obtained by the search using “leukemia” as the keyword is 2, the number of character strings included in the clinical trial item obtained by the narrowed-down search in which the keyword of “xx inhibitor” is further added to “leukemia” is 1, and the subtype ratio is ½=0.5 (that is, 50%).

In this example, the fact that the subtype ratio of the cluster including “ECOG is 0 to 3” is high indicates that, when the selection criterion related to the ECOG is included in the clinical trial item of the drug for the leukemia, a frequency at which the selection criterion is included as the clinical trial item is also high in the clinical trial having the selection criterion related to the xx inhibitor of the drug for the leukemia. On the other hand, the fact that the subtype ratio of the cluster including “ECOG is 0 to 3” is low indicates that, when the selection criterion related to the ECOG is included in the clinical trial item of the drug for the leukemia, a frequency at which the selection criterion is included as the clinical trial item is low in the clinical trial having the selection criterion related to the xx inhibitor of the drug for the leukemia.

That is, it can be said that the higher the subtype ratio of the cluster is, the higher a frequency at which the clinical trial item of the cluster is adopted in the past clinical trial of the drug having adaptation similar to that of the drug for which the user attempts to conduct the clinical trial. In other words, adaptation of a clinical trial corresponding to a cluster with a high subtype ratio is considered to be similar to the adaptation of the clinical trial that the user attempts to conduct. Therefore, the user can efficiently create a clinical trial conduction plan by preferentially examining whether the clinical trial items of the clusters having a high subtype ratio are to be adopted in the clinical trial to be conducted thereafter.

In step 806, the generated clusters are output in a descending order of the subtype ratio. For example, the generated clusters may be displayed in a list in the descending order of the subtype ratio. In the example in FIG. 10, the subtype ratio of the cluster (that is, the cluster including the character string similar thereto) including the character string “resistance to xx inhibitor” is the highest, the subtype ratio of the cluster including the character string “not subjected to chemotherapy” is the second highest, and similarly, the subtype ratio of the cluster including the character string “ECOG is 0 to 3” and the subtype ratio of the cluster including the character string “normal renal function” are sequentially high. In the example in FIG. 10, a graph indicating a height of the subtype ratio of each cluster is displayed together with the character string representing the cluster.

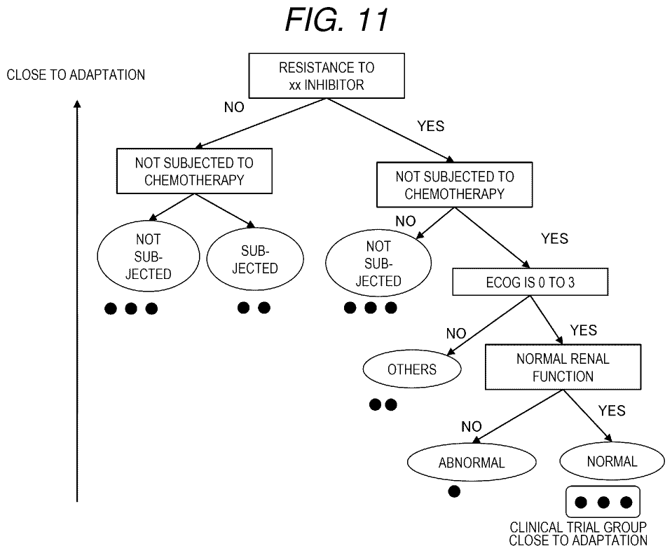

FIG. 11 is a diagram showing a classification of clinical trials output by the similar clinical trial search support system 150 according to the embodiment of the invention.

The similar clinical trial search support system 150 according to the embodiment may classify the clinical trial groups stored in the storage unit 123 based on the clustering results and the calculated subtype ratios of the clusters, and output the results.

In the example in FIG. 10, the clusters “resistance to xx inhibitor”, “not subjected to chemotherapy”, “ECOG is 0 to 3”, and “normal renal function” are generated in the descending order of the subtype ratio. In this case, the similar clinical trial search support system 150 classifies the clinical trials according to whether the clinical trials include the clinical trial item that satisfies a condition “resistance to xx inhibitor” with the highest subtype ratio.

Next, the similar clinical trial search support system 150 classifies clinical trial groups that satisfy the condition “resistance to xx inhibitor” according to whether the clinical trial groups include a clinical trial item that satisfies a condition “not subjected to chemotherapy”. Similarly, the similar clinical trial search support system 150 classifies the clinical trial groups that satisfy the condition “not subjected to chemotherapy” according to whether the condition “ECOG is 0 to 3” is satisfied, and further classifies the clinical trials that satisfy the condition “ECOG is 0 to 3” according to whether the clinical trials include the clinical trial items that satisfy the condition of having “normal renal function”.

FIG. 11 shows an example in which a result of the above-described classification is displayed in a form of a decision tree. Although not shown in FIG. 11, the clinical trial groups that do not satisfy the above-described conditions are also classified based on whether the dominant conditions are satisfied, and the results are displayed.

In the example in FIG. 11, a clinical trial group that satisfies all of the above-described conditions is the clinical trial group that is closest to the adaptation of the drug for which the user attempts to conduct the clinical trial. The number of black circles in FIG. 11 indicates the number of clinical trials included in the clinical trial group satisfying each condition. In this example, it can be said that the more clinical trials satisfy the conditions corresponding to nodes in an upper layer, the more similar the adaptation is to the adaptation of the drug for which the user attempts to conduct the clinical trial. In other words, the more the clinical trials do not satisfy the conditions corresponding to the nodes in the upper layer, the further the adaptation is from the adaptation of the drug for which the user attempts to conduct the clinical trial.

Therefore, when creating a clinical trial conduct plan, the user can use the information shown in FIG. 11 to determine a priority of the clinical trial groups to be referred to by, for example, referring to the clinical trial items of the clinical trial groups satisfying all the above-described conditions first, and then referring to the clinical trial items of the clinical trial groups satisfying all the three conditions in the upper layer and not satisfying the condition in the lowest layer. Accordingly, efficient clinical trial conduct plan creation is supported.

The system according to the embodiment of the invention may be formed as follows.

1. A similar clinical trial search support system includes: a processing unit (for example, the processing unit 122); and a storage unit (for example, the storage unit 123). The storage unit stores clinical trial information (for example, information stored in the clinical trial information storage unit 127) that associates identification information of each of a plurality of clinical trials conducted in the past with a content of the clinical trial, and clinical trial item information (for example, information stored in the clinical trial item storage unit 128) that associates the identification information of the clinical trial with one or more clinical trial items in which a setting item of the clinical trial is described in a character string. The processing unit searches the clinical trial information based on a first search item (for example, “leukemia”) to acquire the identification information of one or more clinical trials as a first search result, adds a second search item (for example, “xx inhibitor”) to the first search item to search the clinical trial information, so as to acquire identification information of the one or more clinical trials as a second search result (for example, steps S801 and S802), acquires a plurality of the clinical trial items corresponding to the identification information of the one or more clinical trials acquired as the first search result from the clinical trial item information (for example, step S803), clusters the plurality of acquired clinical trial items to generate a plurality of clusters (for example, step S804), calculates, for each cluster, a ratio (for example, a subtype ratio) of the number of the clinical trial items corresponding to the identification information of the clinical trial acquired as the second search result in the clinical trial items classified into the cluster (for example, step S805), and outputs information (one character string included in the cluster) for specifying the cluster, and the ratio calculated for the cluster (for example, step S806).

Accordingly, a polygonal analysis of the past clinical trial information focusing on similarity to adaptation of a development drug is supported.

(2) In the above-described (1), the storage unit stores medical organism term information (for example, information stored in the medical organism term storage unit 131) including at least one of a medical term and an organism term, and the processing unit converts a word string constituting a character string of the acquired clinical trial items into a vector string of words, assigns a predetermined weight to a vector of a word included in the medical organism term information among vectors constituting the vector string of words, converts the vector string of words into a vector representation of a sentence (for example, step S903), and clusters the plurality of clinical trial items using the vector representation of a sentence.

Accordingly, similarity of adaptation of the clinical trial is appropriately determined.

(3) In the above-described (2), the medical organism term information includes information indicating a relationship between a term of a dominant concept and a term of a subordinate concept, and a weight assigned to a vector of the term of a subordinate concept is greater than a weight assigned to a vector of the term of a dominant concept.

Accordingly, the similarity of the adaptation of the clinical trial is appropriately determined.

(4) In the above-described (1), the processing unit outputs information for specifying the clusters in a descending order of the ratio (for example, FIG. 10).

Accordingly, useful information for analyzing the clinical trial information is efficiently provided to the user.

(5) In the above-described (1), the processing unit classifies the plurality of clinical trials included in the clinical trial information according to whether the setting item in the clinical trial items of the clusters are satisfied in a descending order of the ratio, and outputs information having a tree structure indicating a result of classifying the plurality of clinical trials (for example, FIG. 11).

Accordingly, useful information for analyzing the clinical trial information is efficiently provided to the user.

(6) In the above-described (1), the content of the clinical trial included in the clinical trial information includes at least two of a phase (for example, the phase 302) of the clinical trial, a drug (for example, the clinical trial drug 304) targeted in the clinical trial, an effect (for example, the effect 303) of the drug, and an action mechanism (for example, the action mechanism 305) of the drug.

Accordingly, the clinical trial information is appropriately analyzed.

(7) In the above-described (1), the setting item of the clinical trial included in the clinical trial item information includes a selection criterion or an exclusion criterion for a subject of the clinical trial (for example, the item 403, the class 404, and the content 405).

Accordingly, the clinical trial information is appropriately analyzed.

The invention is not limited to the above-described embodiments, and includes various modifications. For example, the above-described embodiments are described in detail for a better understanding of the invention, and the invention is not necessarily limited to those including all the configurations described above. A part of a configuration according to one embodiment can be replaced with a configuration according to another embodiment, and a configuration according to one embodiment can also be added to a configuration according to another embodiment. A part of the configuration of each embodiment may be added to, deleted from, or replaced with another configuration.

A part or all of configurations, functions, processing units, processing methods, and the like described above may be implemented by hardware by, for example, designing with an integrated circuit. In addition, the configurations, functions, and the like described above may be implemented by software by a processor interpreting and executing a program for implementing each function. Information such as a program, a table, and a file for implementing each function can be stored in a storage device such as a nonvolatile semiconductor memory, a hard disk drive, and a solid state drive (SSD), or a computer-readable non-transitory data storage medium such as an IC card, an SD card, and a DVD.

Control lines and information lines indicate what is considered to be necessary for description, and not necessarily all control lines and information lines are shown on a product. Actually, almost all components may be considered to be connected to one another.

What is claimed is:

1. A similar clinical trial search support system comprising:
a processing unit; and
a storage unit, wherein
the storage unit stores clinical trial information that associates identification information of each of a plurality of clinical trials conducted in the past with a content of the clinical trial, and clinical trial item information that associates the identification information of the clinical trial with one or more clinical trial items in which a setting item of the clinical trial is described in a character string, and
the processing unit
searches the clinical trial information based on a first search item to acquire the identification information of one or more clinical trials as a first search result,
adds a second search item to the first search item to search the clinical trial information, so as to acquire identification information of the one or more clinical trials as a second search result,
acquires a plurality of the clinical trial items corresponding to the identification information of the one or more clinical trials acquired as the first search result from the clinical trial item information,
clusters the plurality of acquired clinical trial items to generate a plurality of clusters,
calculates, for each cluster, a ratio of the number of the clinical trial items corresponding to the identification information of the clinical trial acquired as the second search result in the clinical trial items classified into the cluster, and
outputs information for specifying the cluster, and the ratio calculated for the cluster.

2. The similar clinical trial search support system according to claim 1, wherein
the storage unit stores medical organism term information including at least one of a medical term and an organism term, and
the processing unit
converts a word string constituting a character string of the acquired clinical trial items into a vector string of words,
assigns a predetermined weight to a vector of a word included in the medical organism term information among vectors constituting the vector string of words,
converts the vector string of words into a vector representation of a sentence, and
clusters the plurality of clinical trial items using the vector representation of a sentence.

3. The similar clinical trial search support system according to claim 2, wherein
the medical organism term information includes information indicating a relationship between a term of a dominant concept and a term of a subordinate concept, and
a weight assigned to a vector of the term of a subordinate concept is greater than a weight assigned to a vector of the term of a dominant concept.

4. The similar clinical trial search support system according to claim 1, wherein
the processing unit outputs information for specifying the clusters in a descending order of the ratio.

5. The similar clinical trial search support system according to claim 1, wherein
the processing unit classifies the plurality of clinical trials included in the clinical trial information according to whether the setting item in the clinical trial items of the clusters are satisfied in a descending order of the ratio, and
outputs information having a tree structure indicating a result of classifying the plurality of clinical trials.

6. The similar clinical trial search support system according to claim 1, wherein
the content of the clinical trial included in the clinical trial information includes at least two of a phase of the clinical trial, a drug targeted in the clinical trial, an effect of the drug, and an action mechanism of the drug.

7. The similar clinical trial search support system according to claim 1, wherein
the setting item of the clinical trial included in the clinical trial item information includes a selection criterion or an exclusion criterion for a subject of the clinical trial.

8. A similar clinical trial search support method executed by a computer system, the computer system including a processing unit and a storage unit, the storage unit storing clinical trial information that associates identification information of each of a plurality of clinical trials conducted in the past with a content of the clinical trial, and clinical trial item information that associates the identification information of the clinical trial with one or more clinical trial items in which a setting item of the clinical trial is described in a character string, the similar clinical trial search support method comprising:
searching, by the processing unit, the clinical trial information based on a first search item to acquire the identification information of one or more clinical trials as a first search result;
adding, by the processing unit, a second search item to the first search item to search the clinical trial information, so as to acquire identification information of the one or more clinical trials as a second search result;
acquiring, by the processing unit, a plurality of the clinical trial items corresponding to the identification information of the one or more clinical trials acquired as the first search result from the clinical trial item information;
clustering, by the processing unit, the plurality of acquired clinical trial items to generate a plurality of clusters;
calculating, by the processing unit for each cluster, a ratio of the number of the clinical trial items corresponding to the identification information of the clinical trial acquired as the second search result in the clinical trial items classified into the cluster; and
outputting, by the processing unit, information for specifying the cluster, and the ratio calculated for the cluster.
